(19) United States
(12) Patent Application Publication
(10) Pub. No.: US 2021/0193327 A1
(43) Pub. Date: Jun. 24, 2021

(54) Reducing Readmission Risk Through Co-Existing Condition Prediction

(71) Applicant: CERNER INNOVATION, INC., Kansas City, KS (US)

(72) Inventors: Revathi Sankar, Chennai (IN); Gopika Chenganikattu, Near Hosa Road Junction (IN)

(21) Appl. No.: 16/719,475

(22) Filed: Dec. 18, 2019

(51) Int. Cl. G16H 50/70; G16H 10/60; G06F 17/18; G06F 17/15; G06F 3/0482

(52) U.S. Cl. CPC G16H 50/70; G16H 10/60; G06F 3/0482; G06F 17/15; G06F 17/18

(57) ABSTRACT

Methods, systems, and computer-storage media are provided for determining the probability of readmission of an individual to a facility after a first admission. Medical data elements are identified that are associated with a first admission and at least one readmission for at least one individual at a facility over a predetermined period. Sequential pattern analysis is performed to determine correlation clusters between two or more conditions. Additionally, multivariate logistic regression may be performed to further support the correlation clusters determined. Based on the analysis, a prediction is generated and communicated to a first user regarding the risk of readmission of an individual and interventional treatments are proposed to decrease the risk of readmission based on the prediction comprising the correlation cluster.

### BACKGROUND

Reducing the risk of a patient being readmitted to a facility, such as a hospital, after a first admission is constant challenge present in healthcare industry. Time and time again, individuals who are admitted to a facility for one condition find themselves returning for readmission for either the same or other co-existing conditions. These high rates of readmission may negatively impact the patient's care experience and are an indication of the lack of thorough and proactive healthcare management. Due to the complexity of healthcare management in today's age, healthcare providers are limited, at times, in their capability to treat individuals for more than the condition presented at admission to the facility. Healthcare providers are limited by factors such as time, resources, and limitations imposed by insurance and facilities, which result in the healthcare provider solely focusing on addressing the current condition for an admission without having the time or ability to determine whether there are other co-existing conditions that should be managed for the individual in order to reduce or prevent a potential readmission to the facility in the future. Co-existing conditions may be present that are either related to or have correlations to a primary condition being treated that are overlooked. In fact, research has shown that there are categories of co-existing conditions that have proven correlations with one another for an individual.

The reduction of the risk of readmission occurring for an individual is a priority for those involved in the individual's medical care including the individual themselves, medical professionals, locations of treatment (hospitals, acute care facilities, etc.), and insurance companies. Evidence indicates an increasing demand for medical management of preventable readmissions to reduce medical care costs and improve the medical status of individuals at risk. As such, identification of conditions that have correlations with one another to determine the readmission risk is critical for more efficient and valuable management of medical care. Admissions that may be preventable through effective medical management may include hospitalizations, emergency department visits, admission to an acute care facility, and admission to an inpatient rehabilitation facility.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

Potentially preventable readmissions may be avoided for individuals for a variety of medical conditions subsequent to a first admission occurring. To accomplish a decrease in readmissions, it is useful to predict co-existing conditions that result in readmission for individuals based on data analysis. Data indicates that there are multiple conditions that have proven correlations with each other and can co-exist for a patient. For example, data from the Centers for Disease Control (CDC) indicate that 49% of adults diagnosed with heart disease are also diagnosed with arthritis, and 47% of adults with diabetes are also diagnosed with arthritis. From a clinical or functionality perspective, these conditions do not have the same root cause or origin. However, according to the data analyzed, these pairs of conditions seem to have a correlation and co-exist in individuals. As such, identification of correlated conditions during a first admission can act as a preventive mechanism to significantly improve the quality of care and reduce the risk of readmission to a facility, such as a hospital. When a healthcare provider is provided with insight into correlated conditions relevant to an individual's condition for admission early on (e.g., at the time of diagnosis of a primary condition associated with the admission of an individual to a hospital), it helps the healthcare provider make the appropriate interventions for the individual ahead of time to decrease the chances that the individual will be readmitted due to the predicted co-existing condition.

Readmissions are costly for both the healthcare system and the individual. For example, the Center for Medicare and Medicaid Services (CMS) monitors readmission rates, and a facility where several readmissions occur may receive a lower level of reimbursement from a payer when the patient is readmitted. In some instances, the cost of readmission for an individual falls entirely on the facility, thereby significantly increasing the cost of healthcare for the facility when a readmission occurs. Data indicates that the rates of readmission to facilities are high and that one in five Medicare beneficiaries is hospitalized within 30 days of a hospital discharge, resulting in costs of over $15 billion annually. As a result, Medicare has implemented a readmission reduction program aimed to reduce unnecessary readmission of patients. Further, several studies have shown a significant portion of the readmissions could be potentially avoided if processes were implemented predicting the chances of readmission based on the determined correlations between co-existing conditions that result in readmission, thereby allowing a healthcare provider to proactively implement preventive treatment to decrease the risk of readmission. As such, predicting the factors for readmission may significantly lower the healthcare cost and improve the quality of care by taking appropriate interventions ahead of time.

While there have been previous attempts to determine the risk of readmission to a facility, many of the methodologies implemented do not accurately capture the risk of readmission by prediction of co-existing conditions that results in readmission. Some approaches rely on human clinical acumen in determining whether a patient is at risk for readmission. However, it has been found that clinicians often lack the ability to accurately identify patients at risk for readmission. Moreover, these approaches are not driven by quantitative data, and lack of a data-driven approach to quantifying a risk for readmission may inhibit the healthcare industry from realizing increases in efficiency as evidenced by, for example, reductions in readmission rates. Further, many of the previous approaches were not customizable to a facility's needs and were instead focused at a population level.

At a high level, the present disclosure identifies a predetermined number of reasons associated with a first admission and at least one readmission for at least one individual over a predetermined time period (e.g., the top 10 reasons for readmission over the last 10 years) for a given facility. Using the data identified, a sample size is established and sequential pattern analysis is performed to determine a correlation cluster between two or more conditions to generate a prediction regarding readmission risk. The prediction indicates the presence of a correlation between a first condition (e.g., arthritis) and a second condition (e.g., diabetes) and readmissions to the facility in the past. By identifying the risk of a readmission, preventative and interventional care management can be utilized to reduce the occurrence and risk of the readmission to a facility such as a hospital, emergency department, acute care facility, and inpatient rehabilitation facility. The result of identifying the risk for readmission based on determined correlation clusters will lead to a decrease in spending on the medical care of an individual as well as an improved care experience for the individual.

The present invention targets a preselected population, which may be identified by a computer system or by medical professionals, and determines a correlation cluster between two or more conditions based on a first admission and at least one readmission in order to potentially prevent or reduce the risk of the readmission occurring. Target populations may include individuals who are hospitalized, are at risk for acute hospitalization, suffer from chronic diseases which require on-going management (e.g., diabetes, heart failure), and multi-disease, multi-complication individuals (e.g., renal failure, individuals who have received transplants, cancer patients).

Additionally, the present disclosure discusses the use of sequential analysis to predict the probability of a second condition occurring based on a determined correlation cluster. The identification of potential co-existing conditions occurs at an early point during the individual's admission (e.g., upon diagnosis with a primary condition), which allows proposed interventions to be timely initiated in order to reduce the individual's risk for readmission. The result is an overall decrease in healthcare spending for the patient as well as an improved care experience for the patient. This will improve the quality of care, increase earlier identification of potential co-existing conditions that could result in readmissions, and provide preventive healthcare services to the individuals, thereby reducing the readmission rates and healthcare spending. As a result, satisfaction for the entire population regarding healthcare will also improve.

Aspects herein describe computer-storage media, computerized methods, and computing systems that determine the probability of readmission of an individual to a facility after a first admission. A computer system contains medical data elements for a pre-selected population that are stored in an electronic medical record store. A computer server, at the computer healthcare system, is coupled to the electronic medical record store and programmed to access the electronic medical record store to identify medical data elements associated with a predetermined number of conditions that are associated with a first admission and at least one readmission to a facility for at least one individual over a predetermined time period. Then medical data elements identified are then analyzed to identify a sample size. Once the sample size is established, the system performs a first sequential pattern analysis on the medical data elements to determine a correlation cluster between two or more conditions related to the first admission and the at least one readmission. In response to determining the correlation cluster, the system generates and communicates to a first user, via a user interface, a prediction for readmission for an individual based on the determined correlation cluster.

As well, aspects herein are also directed to calculating one or more of a support percentage, a confidence level, and a correlation coefficient on the correlation cluster to further substantiate the correlation cluster findings. Additionally, a multivariate logistic regression on the correlation cluster is performed to determine a regression coefficient and an odds ratio which determine the impact of various parameters on the occurrence of the correlation cluster. Utilizing this information, the system will generate and communicate to a first user, via a user interface, a prediction for readmission for a first individual based on the determined correlation cluster.

Further, as described herein, when an individual is admitted to a facility and diagnosed with a condition previously identified as one of the conditions associated with a first admission and at least one subsequent readmission to a facility for at least one individual over a predetermined time period, the healthcare provider can receive an indication, within an electronic medical record (EMR) that the condition is one of the conditions associated with the determined correlation clusters. Based on this indication, the system can provide the healthcare provider with a prediction that suggests interventions to reduce the risk of readmission. The healthcare provider may either accept the suggested intervention or reject it. When the healthcare provider accepts one or more interventional suggestions, the system receives feedback indicating whether the treatment was effective in preventing the readmission of the individual for one of the co-existing conditions in the correlation cluster. Over time, clinical protocols may also be updated for a facility based on success in reducing readmission for individuals based on the predictions comprising the correlation clusters data.

### BRIEF DESCRIPTION OF THE DRAWINGS

Embodiments are described in detail below with reference to the attached drawings figures, wherein:

FIG. 1 is a block diagram of an exemplary computing environment suitable to implement embodiments of the present invention;

FIG. 2 is an exemplary system architecture suitable to implement embodiments of the present invention;

FIG. 3 illustrates an exemplary process of determining a correlation cluster comprising two or more conditions and a prediction regarding the probability of readmission of an individual to a facility;

FIG. 4 illustrates an exemplary sequential pattern analysis conducted on medical data elements resulting in two correlation clusters;

FIG. 5 illustrates a multivariate logistic regression conducted on two correlation clusters from FIG. 4; and

FIG. 6 is a flow diagram depicting an exemplary method of executing embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Embodiments of the present invention are directed to methods, systems, and computer-storage media for computer-based medical information users to monitor an individual's risk for a second event to occur subsequent to a first event based on an analysis of pre-selected medical data elements. Following a first event, a server automatically accesses an electronic medical record store on a predetermined schedule to sample a pre-selected set of medical data elements. A logistic regression analysis is completed on the pre-selected set of medical data elements to generate a second risk score indicating the degree of risk that the second event will occur.

As used herein, the term facility may be any facility which provides healthcare to an individual such as, but not limited to, a hospital, acute care facility, rehabilitation facility, urgent care facilities, and the like.

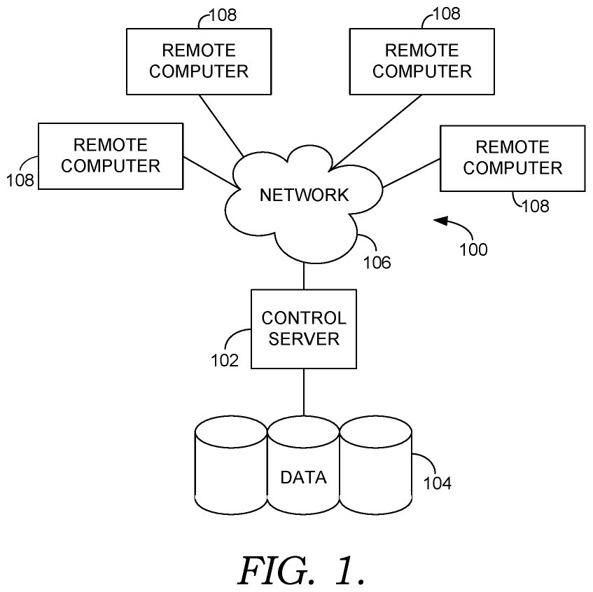

An exemplary computing environment suitable for use in implementing embodiments of the present invention is described below. FIG. 1 is an exemplary computing environment (e.g., medical-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. It will be appreciated by those having ordinary skill in the art that the connections illustrated in FIG. 1 are also exemplary as other methods, hardware, software, and devices for establishing a communications link between the components, devices, systems, and entities, as shown in FIG. 1, may be utilized in the implementation of the present invention. Although the connections are depicted using one or more solid lines, it will be understood by those having ordinary skill in the art that the exemplary connections of FIG. 1 may be hardwired or wireless, and may use intermediary components that have been omitted or not included in FIG. 1 for simplicity's sake. As such, the absence of components from FIG. 1 should not be interpreted as limiting the present invention to exclude additional components and combination(s) of components. Moreover, though devices and components are represented in FIG. 1 as singular devices and components, it will be appreciated that some embodiments may include a plurality of the devices and components such that FIG. 1 should not be considered as limiting the number of a device or component.

The present technology might be operational with numerous other special-purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be operational and/or implemented across computing system environments such as a distributed or wireless “cloud” system. Cloud-based computing systems include a model of networked enterprise storage where data is stored in virtualized storage pools. The cloud-based networked enterprise storage may be public, private, or hosted by a third party, in embodiments. In some embodiments, computer programs or software (e.g., applications) are stored in the cloud and executed in the cloud. Generally, computing devices may access the cloud over a wireless network and any information stored in the cloud or computer programs run from the cloud. Accordingly, a cloud-based computing system may be distributed across multiple physical locations.

The present technology might be described in the context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of non-transitory computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media.

The control server 102 might operate in a computer network 106 using logical connections to one or more remote computers 108. Remote computers 108 might be located at a variety of locations including operating systems, device drivers, and medical information workflows. The remote computers might also be physically located in traditional and nontraditional medical care environments so that the entire medical community might be capable of integration on the network. The remote computers might be personal computers, servers, routers, network PCs, peer devices, other common network nodes, or the like and might comprise some or all of the elements described above in relation to the control server. The devices can be personal digital assistants or other like devices. Further, remote computers may be located in a variety of locations including in a medical or research environment, including clinical laboratories (e.g., molecular diagnostic laboratories), hospitals and other inpatient settings, veterinary environments, ambulatory settings, medical billing and financial offices, hospital administration settings, home healthcare environments, and clinicians' offices. Medical professionals may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like. The remote computers 108 might also be physically located in nontraditional medical care environments so that the entire medical community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a microphone (e.g., voice inputs), a touch screen, a pointing device (commonly referred to as a mouse), a trackball, or a touch pad. Other input devices comprise satellite dishes, scanners, or the like. Commands and information might also be sent directly from a remote medical device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

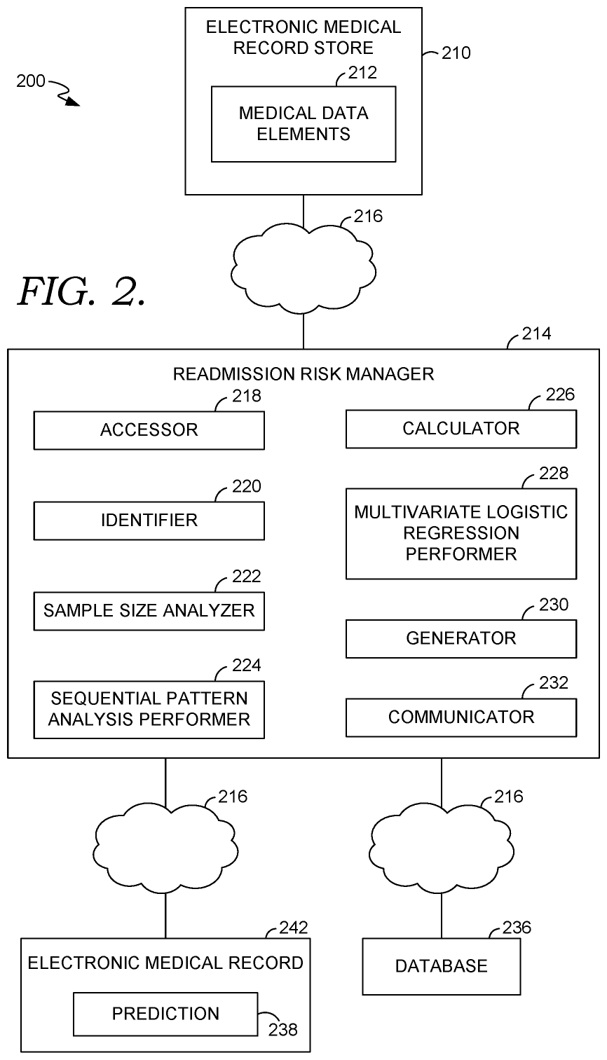

Turning now to FIG. 2, an exemplary computing system 200 is depicted. The computing system 200 (hereinafter “system”) is merely an example of one suitable computing system and is not intended to suggest any limitation as to the scope of use or functionality of embodiments of the present invention. Neither should the system 200 be interpreted as having any dependency or requirement related to any single component or combination of components illustrated herein.

In some embodiments, one or more of the illustrated components may be implemented as a stand-alone application. The components described are exemplary in nature and in number and should not be construed as limiting. Any number of components may be employed to achieve the desired functionality within the scope of the embodiments hereof. Further, components may be located on any number of servers.

The system 200 includes an electronic medical record store 210, a readmission risk manager 214, an electronic medical record 242, and a database 236. The electronic medical record store 210 is comprised of medical data elements 212. The medical data elements 212 stored in the electronic medical record store 210 may comprise, in exemplary aspects, medication information, vital sign information, demographic information, laboratory and/or procedure values and results, medical history (symptoms, diagnoses, and the like), medication history, medical procedure history, treatment history, number of readmissions and causes for readmission, social determinants (health literacy, behavioral factors, support network, and the like), assessment information for the individual, and any other pertinent medical data monitored by a healthcare system.

In this embodiment, the readmission risk manager 214 is comprised of an accessor 218, an identifier 220, a sample size analyzer 222, a sequential pattern analysis performer 224, a calculator 226, a multivariate logistic regression performer 228, a generator 230, a communicator 232, and a receiver 234. In this aspect, the readmission risk manager 214 is comprised of eight subcomponents (listed above). However, in other aspects, the readmission risk manager 214 may be comprised of more or less components and any and all variations are contemplated herein. It will be appreciated that some or all of the subcomponents of the readmission risk manager 214 may be accessed via the network 216 and may reside on one or more devices. The readmission risk manager 214 can perform risk surveillance on some or all of the individuals in a pre-selected population and is in communication with the electronic medical record store 210, the database 236, and the electronic medical record 242 via the network 216. Additionally, in this exemplary aspect, the electronic medical record 242 includes a prediction 238.

In aspects, the electronic medical record store 210 stores medical data elements 212 for individuals that have had at least a first admission to a facility take place. A first admission includes, but is not limited to, hospitalization, emergency department visits, and admission to inpatient care facilities or rehabilitation facilities. The medical data elements 212 may be supplied by the electronic medical record (EMR) 242 of the individual. The EMR 242 of an individual may comprise, for instance, medical records from the healthcare professionals managing the individual's medical care (e.g., primary care, specialists, etc.), pharmacy records, hospital admission records, and the like. Additionally, the EMR 242 also comprises information such as race, ethnicity, age, gender, geographical locations, family history data, and social determinants of health, which all impact the healthcare management and overall health condition of individuals. When a first admission takes place for the individual, medical records stored in the individual's EMR 242 may be communicated to the electronic medical record store 210 and/or the readmission risk manager 214. If subsequent admissions take place, the medical records stored in the individual's EMR 242 may be further communicated to the electronic medical record store 210 and/or the readmission risk manager 214. As such, the medical data elements 212 found in the electronic medical record store 210 may include data related to both a first admission and subsequent readmissions, which may further comprise data such as diagnosed conditions.

To begin the determination of the probability of readmission, the accessor 218 is configured to access the electronic medical record store 210. The accessor 218 accesses the electronic medical record store 210 so that medical data elements 212 that are associated with a predetermined number of conditions that are associated with a first admission and at least one readmission to a facility for at least one individual over a predetermined period of time can be identified. The number of conditions associated with a first admission and at least one readmission may vary based on the facility.

Once the accessor has accessed the electronic medical record store 210, the identifier 220 will identify the predetermined number of conditions associated with the first admission and at least one readmission over a predetermined time period. For example, in a large hospital system located in a diverse urban area, there may be several conditions that have been found to be the top reasons for readmission at the facility over a predetermined time period. The predetermined number of conditions identified by the identifier 220 may range from two to hundreds or thousands of conditions. The predetermined time period may be, for example, the last 10 years. As such, the accessor 218 will access the electronic medical record store 210 so that the identifier 220 can identify medical data elements for the last 10 years. The identifier 220 may identify, for example, the top 10 conditions for a first admission and at least one readmission to the facility over the last 10 years. Examples of conditions that may comprise the top ten reasons or conditions include, but are not limited to, diabetes, arthritis, heart disease, and strokes.

After the identifier 220 has identified medical data elements associated with a predetermined number of conditions that are associated with a first admission and at least one readmission to a facility for at least one individual, the sample size analyzer 222 analyzes the medical data elements identified to establish a sample size. The sample size established by the sample size analyzer 222 depends on a variety of factors. If the medical data elements identified by the identifier 220 are for a large hospital system, then the number of medical data elements analyzed will be larger than a sample size for a small community hospital. However, at minimum, a large enough sample size must be established to run a sequential pattern analysis to establish correlation pairs of two or more conditions. As such, in some aspects, there must be a minimum of 5000 medical data elements available to conduct the sequential pattern analysis.

Once the sample size is established by the sample size analyzer 222, the sequential pattern analysis performer 224 performs a first sequential pattern analysis on the medical data elements 212 to determine a correlation cluster between two or more conditions related to the first admission and the at least one readmission. The sequential analysis performed is a method of statistical evaluation used to study the strength of a relationship between two, numerically measured, continuous variables (e.g., height and weight). Sequential analysis is utilized to show that when the medical data elements 212 in the sample are analyzed over the predetermined time period (e.g., 10 years), when condition A occurs, there is a frequent occurrence of condition B occurring as well and vice versa. The correlation clusters determined by the sequential pattern analysis performer 224 result in pairs or clusters of conditions (two or more) which have been found to have proven correlations with one another. For example, a correlation cluster may be determined by the sequential pattern analysis performer 224 between arthritis and diabetes. As such, the sequential pattern analysis performer 224 determines that, in the sample analyzed, when an individual is diagnosed with arthritis there is a frequent occurrence of diabetes as well. As such, when these co-existing conditions exist, the medical data elements 212 analyzed indicate that there is a greater risk of readmission after a first admission for one of the conditions in the correlation cluster comprising arthritis and diabetes. While this example indicates a correlation cluster between two conditions, it is contemplated that, based on the analysis of the medical data elements 212 and the sequential pattern analysis performed, there may also be correlation clusters determined between more than two conditions (e.g., correlation cluster found between diabetes, arthritis, and heart disease).

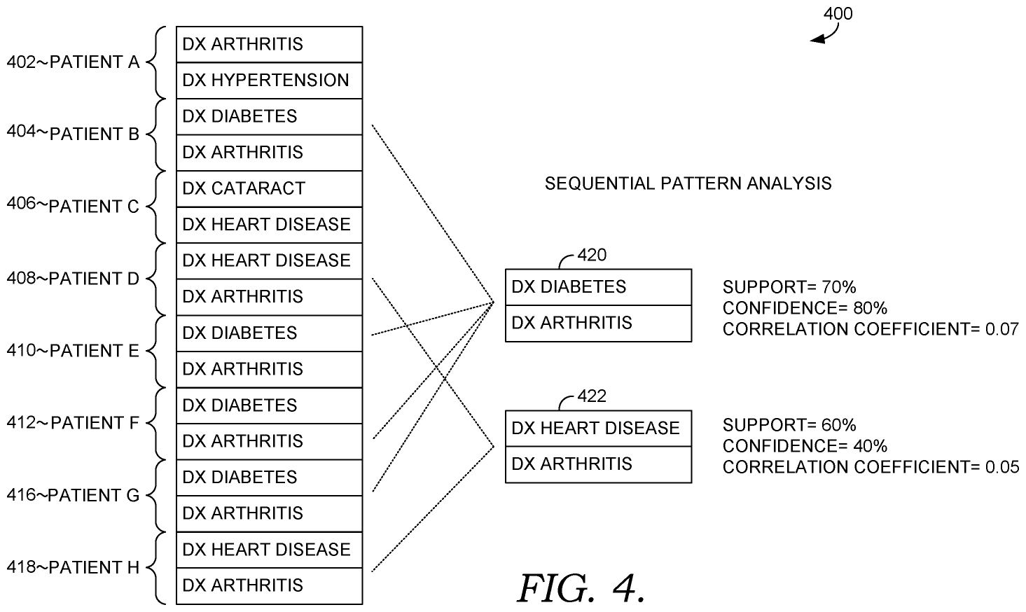

In order to further support such findings, the calculator 226 further calculates a support percentage for each correlation cluster determined. The support percentage indicates the percent of time where the two or more conditions are co-existing. For example, when the calculator 226 determines a correlation cluster between a diabetes diagnosis and arthritis diagnosis, the calculator 226 may calculate a support percentage of 70% (shown in FIG. 4), meaning that the sequential pattern analysis performer 224 determined that, in the past, there was a 70% occurrence of the correlation cluster. As such, as shown in FIG. 4, the support percentage of 70% for the correlation cluster between diabetes and arthritis indicates that, in the past, there has been a 70% occurrence of diabetes and arthritis over the 10 year time period.

Additionally, the calculator 226 may also calculate a correlation coefficient for the correlation cluster determined. Continuing with the diabetes and arthritis correlation cluster example, the calculator 226 may determine that the correlation coefficient between the two conditions is 0.07, which indicates the linear dependence of the two variables, arthritis and diabetes. The correlation coefficient measures the strength of the relationship between two conditions or the degree of association between the two variables. In other words, it measures the probability that the same conditions can occur in the same individual. Complete correlation between two variables is expressed by either +1 or −1. When one variable increases as the other increases, the correlation is positive and when one decreases as the other increases, it is negative. As shown, in the example discussed in FIG. 4, the probability that diabetes and arthritis can occur in the same individual is 0.07, indicating that there is a fairly strong correlation between diabetics and arthritis.

Further, the calculator 226 may also calculate a confidence percentage, which indicates the probability that the same set of conditions will occur in the future. In the diabetes and arthritis correlation cluster example (shown at 420 in FIG. 4), there is an 80% confidence percentage that the same set of conditions (e.g., diabetes and arthritis) will occur together in the future. While the confidence percentage shown in FIG. 4 is shown as 80%, this determination is merely exemplary. The support percentage, correlation coefficient, and confidence percentage are types of additional statistical analysis applied on the data from the sequential analysis to strengthen the support of the correlation clusters determined and the predictions for the correlation between the two or more conditions. Based on these additional calculations, the probability that the correlation cluster 420 comprising diabetes and arthritis will occur together is greater than the probability that the correlation cluster 422 comprising heart disease and arthritis will occur.

In some aspects, once the sequential pattern analysis performer 224 has determined a correlation cluster between two or more conditions related to the first admission and the at least one readmission, the generator 230 will generate a predication for readmission for an individual. The prediction is then communicated by the communicator 232 to a first user via a user interface.

In other aspects, prior to the generator 230 generating a prediction for readmission for an individual, a multivariate logistic regression performer 228 performs a multivariate logistic regression on the data from the correlation cluster. A multivariate logistic regression is performed in order to determine the effect of factors such as age, gender, ethnicity, geography, family history, and social determinants of health on the correlation clusters determined by the sequential pattern analysis performer 224. Social determinant data elements may comprise health literacy, behavioral factors, support network, and any other components which may play a role in the individual's current condition, in the occurrence of the first admission, and in the future regarding general medical management for the individual. These elements may play a role in the risk of readmission for an individual. In exemplary aspects, the multivariate logistic regression performer 228 may also be configured to assign a weight to one or more of the factors analyzed. For example, family history may be weighted more heavily than the gender of the individual.

Once the multivariate logistic regression is performed by the multivariate logistic regression performer 228, an odds ratio and regression coefficient are determined. An odds ratio is a measure of association between the presence or absence of two properties. The odds ratio is a statistic that quantifies the strength of the association between two events, such as the diagnosis of diabetes with a first admission and a readmission due to an arthritis diagnosis. Stated another way, the odds ratio is defined as the ratio of the odds of A in the presence of B and the odds of A in the absence of B. The odds ratio further substantiates the impact on the conditions by the various parameters, such as age, gender, ethnicity, geography, and family history analyzed in the multivariate logistic regression performed by the multivariate logistic regression performer 228. Further, the multivariate logistic regression performer 228 also generates a regression coefficient which indicates whether there is a positive or negative correlation between each independent variable and the dependent variable. Then, based on the results of the multivariate logistic regression analysis, the generator 230 will generate the prediction 238 for readmission for an individual based on the determined correlation cluster, and the communicator 232 will communicate the prediction 238 to the first user on the first user interface.

As shown in FIG. 2, the prediction 238 may be generated within the electronic medical record 242. As such, when the user is utilizing the EMR to manage the healthcare of an individual, the prediction 238 will appear within the EMR so that the healthcare provider can utilize the predication in their treatment and management of an individual's health. For example, if an individual presents at a facility and is admitted with a condition that falls within one of the correlation clusters determined, the prediction 238 may be presented within the EMR 242 for the healthcare provider to review at the time of admission or shortly thereafter when a diagnosis is made. In other aspects, it is contemplated that the prediction 238 may be provided to a first user via any application that is configured to communicate with the readmission risk manager 214 via the network 216.

Continuing with the previous example, if an individual is admitted to the hospital and is diagnosed with arthritis, which is one of the conditions that fell within the correlation cluster determined, then the system 200 may generate the prediction 238 proactively to the healthcare provider in the EMR 242. Additionally, based on the prediction 238, the EMR may provide the healthcare provider with suggested interventions in order to decrease the risk of readmission for the individual. At the time the suggested interventions are presented to the healthcare provider, the healthcare provider may either accept or reject the proposed interventions. In both instances, feedback regarding the healthcare provider's decision and the outcome of such decision is communicated back to the system 200. If the healthcare provider accepts the suggested interventions, then the feedback will include whether the interventions were successful in preventing a readmission for the individual. Additionally, the prediction 238 comprising the correlation cluster is stored in a database, such as database 236 for future use.

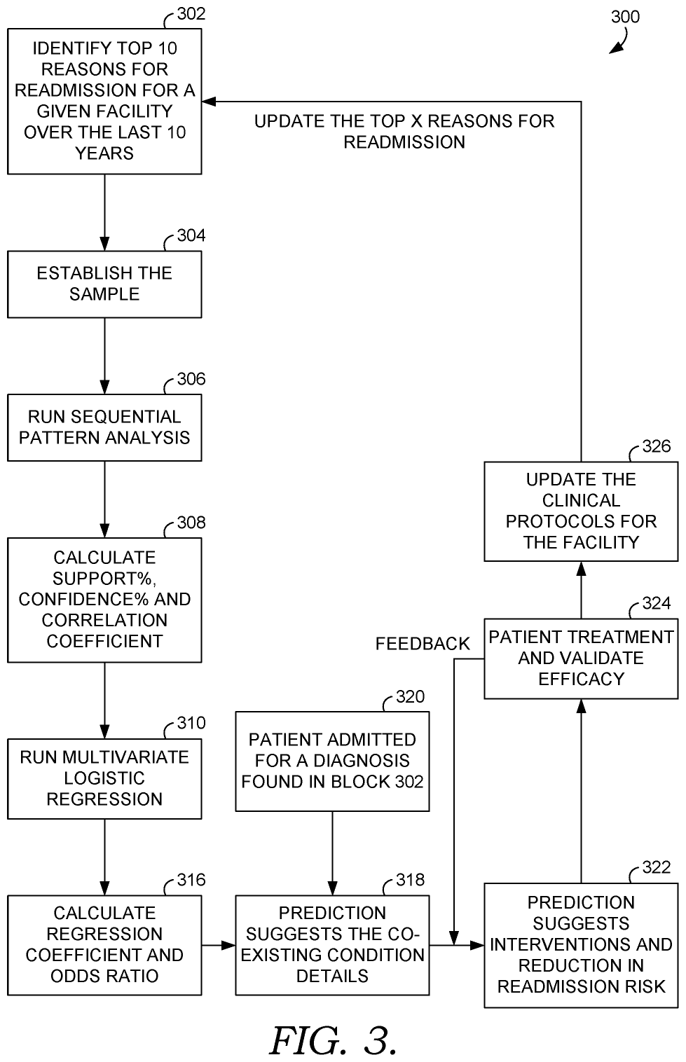

Turning now to FIG. 3, an exemplary process of determining the probability of readmission of an individual to a facility after a first admission and utilizing the determination for interventional treatment is shown. As described, the process begins at block 302 when the accessor 218 accesses the electronic medical record store 210 so that the identifier 220 can identify the predetermined number of reasons for readmission for a facility over a certain time period. As described, the identifier 220 may identify, for example, the top 10 reasons for readmission for a given facility over the last 10 years. In other aspects, the number of reasons may be fewer than 10 or greater than 10 and the number of years may one year or more.

Once the identifier 220 has identified the top 10 reasons for readmission for individuals at a given facility for the last 10 years, the sample size analyzer 222, establishes the sample size at block 304. As mentioned, the sample size may vary based on the size of the facility and the conditions being analyzed, but generally, must be large enough to run the sequential pattern analysis. In some instances the sample size can include a single facility, several facilities, or an entire population. Once the sample size is established, the sequential pattern analysis performer 224 performs the sequential pattern analysis at block 306 to determine a correlation cluster between two or more conditions. The sequential pattern analysis is further supported by the calculation of a support percentage, confidence percentage, and correlation coefficient at block 308 by the calculator 226.

Once the correlation cluster is determined by the sequential pattern analysis performer 224, the multivariate logistic regression performer 228 will perform a multivariate logistic regression at block 310 to take into account the effect of factors such as age, gender, ethnicity, geography, family history, and social determinants of health. The multivariate logistic regression results in the calculation of a regression coefficient and odds ratio by the calculator 226. While the calculator 226 is described as calculating the odds ratio, regression coefficient, support percentage, confidence percentage, and correlation coefficient, it is contemplated that in some aspects, a different and separate component of the readmission risk manager 214 may calculate the regression coefficient and odds ratio at block 316.

Once the sequential pattern analysis and multivariate logistic regression is completed, a prediction 238 is generated at block 318. The prediction 238 comprises the co-existing condition details determined through the analysis at blocks 302-316. As such, the prediction includes the details regarding the correlation clusters determined between the two or more conditions, including the support percentage, confidence percentage, correlation coefficient, odds ratio, and regression coefficient statistics supporting the correlation found between the two or more conditions in the sample size. The prediction 238 may also suggest interventions to reduce the readmission risk at block 322. Such interventions may include potential treatment plans, medications, dietary or lifestyle changes, or therapies that may decrease the likelihood that one or more of the co-existing conditions in the correlation cluster will occur and that individuals will be readmitted to the facility with one of the conditions found in the correlation cluster.

For example, the prediction 238 generated by the generator 230 and communicated to the healthcare provider via the EMR 242 may include medication to prevent diabetes when the correlation cluster determined includes diabetes and arthritis and the individual has had a first admission for either diabetes or arthritis. As such, if an individual is admitted for a diagnosis found in block 302, then at block 320, the healthcare provider may be provided with the prediction 238 at the time of the diagnosis during the first admission for the individual. As such, this presents an opportunity to proactively prevent a readmission or at least reduce the risk of readmission and potentially reduce the risk that other conditions in the correlation cluster occur. Therefore, at the time of the first admission, the healthcare provider is provided with the prediction 238 on the EMR 242 so that they can take such information into account with their treatment planning and try to prevent a readmission due to one of the conditions of the correlation cluster. In some aspects, the system 200 may also display the cost and coverage of the suggested interventions based on the individual's insurance plan or Medicare/Medicaid benefits at block 322. Based on this information, the healthcare provider may choose the intervention that will reduce the cost of healthcare for the individual and facility.

Additionally, the system 200 may intelligently update the prediction 238 presented to a healthcare provider multiple times during the treatment of an individual admitted to a facility based on the course of care for the individual. For example, as multiple diagnoses are established regarding an individual's health while admitted to a facility, the system 200 may generate multiple predictions when additional conditions are diagnosed that are found to exist within correlation clusters determined by the sequential pattern analysis performer 224. The continuous intelligent updating of the system 200 to generate multiple predictions 238 and potential interventions will allow healthcare providers to proactively address potential secondary conditions that could result in readmissions, thereby leading to a further decrease in the number of readmissions at the facility and decreasing the healthcare costs for both the facility, the individual, and insurance companies/Medicare.

If the healthcare provider chooses to utilize the suggested interventions at block 322, then the healthcare provider may choose to treat the patient to reduce the risk of readmission at block 324. In some instances, utilizing the proposed interventions may also prevent the individual from acquiring an additional condition in the correlation cluster. In other instances, the when an individual already presents with signs and symptoms of an additional condition in the correlation cluster, the proposed interventions at block 322 can improve the prognosis and, as a result, either decrease the readmission risk or decrease the length of stay during a readmission through the preventative and proactive care.

Further, such action may validate the efficacy of the prediction 238 based on the correlation clusters determined. The system 200 will receive feedback from the treatment provided so that the system 200 can learn whether or not the suggested interventions were successful in reducing the risk of readmission. Additionally, the system 200 will learn from the accepted interventions and populate more of the successful interventions in future instances with further proven effectiveness.

In aspects, if the healthcare provider dismisses or rejects the proposed interventions presented at block 322, this feedback will be received by the system 200 as well. This will allow the system 200 to learn from the dismissed interventions and further customize suggestions in the future. Ideally, the prediction 238 generated regarding the determined correlation cluster at the time of diagnosis of a first condition will prevent the diagnosis of an additional condition within the correlation cluster.

Further, if the process is effective and the rate of readmission of the conditions found in the correlation cluster decreases, one or more conditions within the correlation cluster may no longer be within the top reasons for readmission to a facility. As such, over time, a given condition may no longer possess a readmission risk. In instances where the interventional treatments are successful, clinical protocols may be updated for the facility at step 326. This may include updating clinical protocols so that the suggested interventions become mandatory interventions or standardized treatments when an individual presents at the facility with a first admission with one or more conditions found to be a part of a determined correlation cluster. When the given condition no longer poses a readmission risk, the system 200 may repeat the process, beginning with identifying a new predetermined set of top reasons for readmission over a more recent time frame. As such, the system 200 is dynamic, self-teaching, and can evolve over time based on the needs of the facility.

Next, FIG. 4, illustrates results from an exemplary sequential pattern analysis 400 performed by the sequential pattern analysis performer 224 on several exemplary medical data elements associated with a predetermined number of conditions that are associated with a first admission and at least one readmission to a facility. As shown in the sequential pattern analysis 400 illustrated, the identifier 220 has identified eight patients that had a first admission and at least one subsequent readmission to a given facility. The sequential pattern analysis 400 looks at how many individuals presented with a particular two or more conditions and how often the combinations occurred within the sample size.

In this example, the top reasons for readmission are shown as arthritis, heart disease, hypertension, cataract, and diabetes. The identifier 220 identified Patients A-H who presented with at least a first admission and a readmission related to one of the determined top reasons for readmission at the facility within the timeframe analyzed (e.g., the last 10 years). Patient A 402 was first diagnosed with arthritis and then diagnosed with hypertension. Patient B 404 was first diagnosed with diabetes and then diagnosed arthritis. Patient C 406 was first diagnosed with cataract and then diagnosed with heart disease. Patient D 408 was first diagnosed with heart disease and then diagnosed with arthritis. Patient E 410, Patient F 412, and Patient G 416 were diagnosed first with diabetes and then arthritis. Patient H 418 was diagnosed first with heart disease and then with arthritis. The order of the diagnoses illustrated in FIG. 4 are exemplary only and the sequential pattern analysis is a bi-directional approach, which means that the pairs of conditions shown for Patients A-H are not restricted as shown. In other words, while Patients B 404, E 410, F 412, and G 416 were diagnosed with diabetes and arthritis, diabetes may be the first condition diagnosed and arthritis may be the second condition or vice versa.

As seen, Patients A-H comprise the sample established by the sample size analyzer 222. Utilizing the sample, the sequential pattern analysis performer 224 performs a first sequential pattern analysis on the medical data elements from Patients A-H and determines two correlation clusters between two or more conditions related to a first admission and at least one readmission found in the sample. A first correlation cluster 420 is determined to exist between diabetes and arthritis. The calculator 226 further calculates a support percentage of 70% for the correlation cluster 420, indicating that this pattern occurs 70% of the time when the diabetes and arthritis are co-existing conditions for an individual. Additionally, the calculator 226 also determined a confidence percentage of 80% and a correlation coefficient of 0.07% for the correlation cluster 420 comprising diabetes and arthritis.

The sequential pattern analysis performer 224 also determined a second correlation cluster 422 from the sample of patients A-H. The second correlation cluster 422 comprises heart disease and arthritis. The support percentage for this correlation cluster was found to be 60%, along with a confidence percentage of 40% and a correlation coefficient of 0.05. As can be seen, in this sample size, there is a higher representation of individuals having the correlation cluster 420 comprising diabetes and arthritis than the correlation cluster 422 comprising heart disease and arthritis. As such, the correlation clusters determined indicate that when conditions comprising the correlation clusters were present in the sample analyzed, these co-existing conditions resulted in a first admission followed by at least one readmission for the individuals analyzed within the sample.

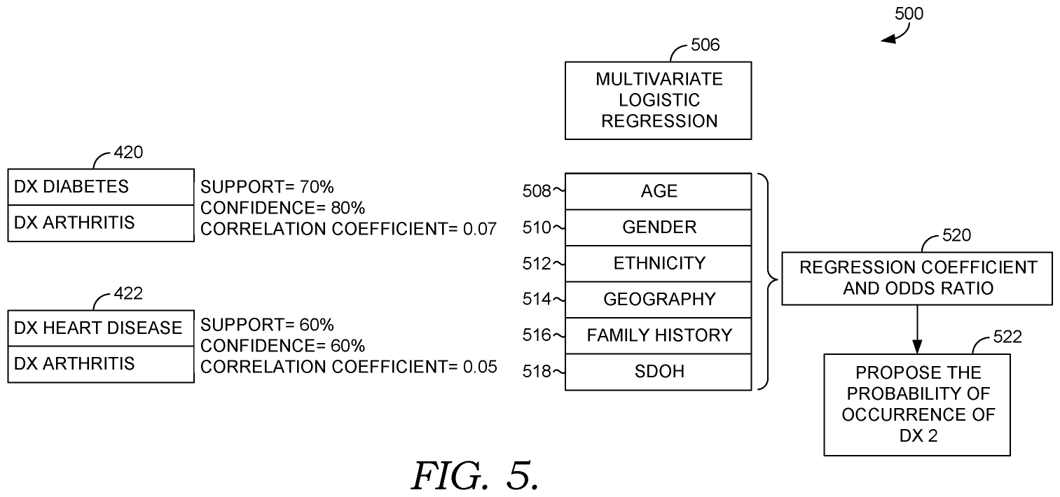

Continuing with FIG. 5, the results of a multivariate logistic regression analysis 500 conducted on the two sets of conditions determined to be a part of correlation clusters 420 and 422 from FIG. 4 are shown. In FIG. 5, the multivariate logistic regression performer 228 performs the multivariate logistic regression utilizing parameter 508-518 to determine the effects on the correlation cluster. As such, the age 508, gender 510, ethnicity 512, geography 514, family history 516, and social determinants of health 518 are factored into the analysis to determine the regression coefficient and odds ratio 522. The multivariate logistic regression performer 228 may apply the effects of only one parameter 508-518, more than one parameter 508-518 or all parameters 508-518 to determine the effects on the correlation cluster.

As shown in FIG. 5, correlation cluster 420 indicates that diabetes and arthritis have a proven correlation to co-exist together for individuals within the sample that were readmitted to the facility at least one time. Based on the determined correlation cluster 420, the multivariate logistic regression performer 228 will determine how each parameter 508-518 is impacting each condition within the cluster. For example, the degree of effect that age 508 has on diabetes and arthritis for correlation cluster 420 is determined. Once the effect of each parameter 508-818 is applied to each condition within the correlation cluster 420 (diabetes and arthritis), a regression coefficient and odds ratio are determined. Previously, prediction analysis included determining the effects of age, gender, ethnicity, and geography. In this disclosure, the use of family history and social determinants of health data from EMR 242, provides a new way to customize the prediction for an individual. This refines the probability of occurrence by adding parameters 508-518 to the analysis completed on the correlation clusters and strengthens the prediction 238 generated to decrease the readmission risk through preventive interventions during the first admission of an individual diagnosed with one or more conditions within the correlation cluster. Additionally, it is contemplated that different parameters may be given different weights.

Based on the regression coefficient and odds ratio 520, the system 200 will propose the probability of the occurrence of the second diagnosis or second condition based on the correlation cluster. In the examples shown in FIGS. 4-5, the multivariate logistic regression will be performed on both the first correlation cluster 420 comprising diabetes and arthritis and a second correlation cluster 422 comprising heart diseases and arthritis. As such, once the multivariate logistic regression is performed, the analysis will indicate the probability that an individual who is diagnosed with diabetes will also be diagnosed with arthritis and an individual who is diagnosed with heart disease will also be diagnosed with arthritis. This data will then be utilized in the prediction 238 presented to a healthcare provider during the treatment of an individual admitted to a facility to reduce the risk of future readmission.

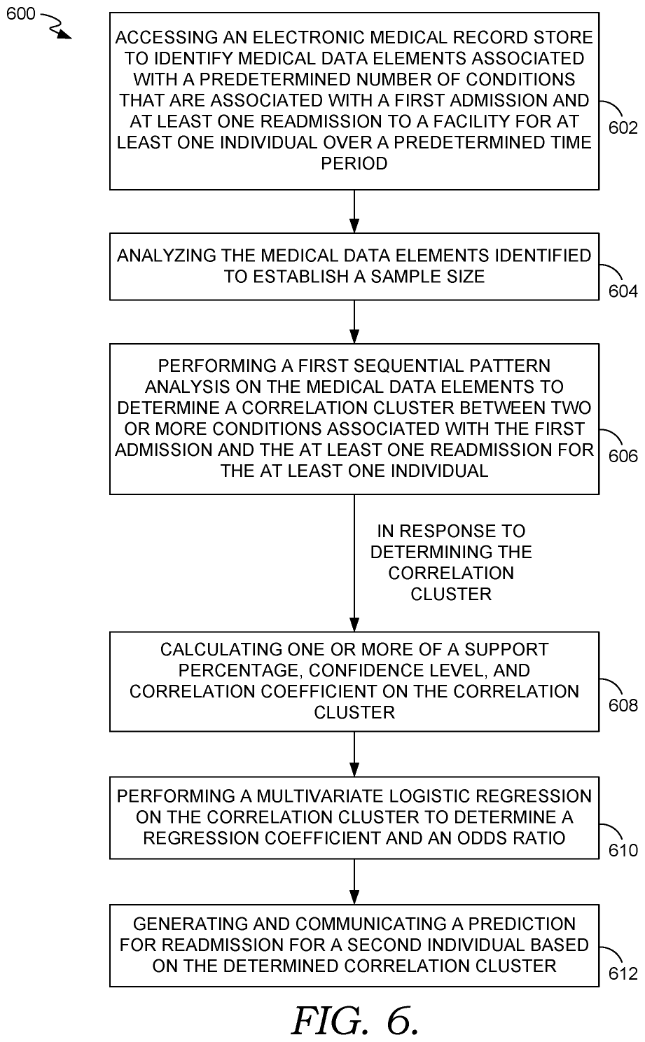

Next, FIG. 6 illustrates a flow diagram depicting an exemplary method 600 of executing the embodiments of the present invention. As shown, the method 600 begins, at block 602, with the accessor 218 accessing the electronic medical record store 210 to identify medical data elements 212 associated with a predetermined number of conditions that are associated with a first admission and at least one readmission to a facility for at least one individual over a predetermined time period. Then, at block 604, the sample size analyzer 222 analyzes the medical data elements identified to establish a sample size. Once the sample size is established, the sequential pattern analysis performer 224 will perform a first sequential pattern analysis on the medical data elements to determine a correlation cluster between two or more conditions associated with the first admission and at least one readmission for the individual at block 606. In response to determining the correlation cluster, the calculator 226 will calculate one or more of a support percentage, confidence level, and correlation coefficient on the correlation cluster at block 608. Then, the multivariate logistic regression performer 228 performs a multivariate logistic regression on the correlation cluster to determine a regression coefficient and an odds ratio at block 610. Based on the findings, the generator 230 will generate a prediction 238 and the communicator 232 will communicate the prediction 238 regarding the co-existing conditions in the correlation cluster and readmission for a first individual. The notification to a the healthcare provider indicating that the individual is at risk for a readmission for treatment for one of the conditions existing within the correlation clusters will trigger care management plans that will prevent or reduce the chances of the readmission from occurring.

The present invention has been described in relation to particular embodiments, which are intended in all respects to be illustrative rather than restrictive. Further, the present invention is not limited to these embodiments, but variations and modifications may be made without departing from the scope of the present invention.

What is claimed is:

1. A dynamic system useful in a computer healthcare system to determine the probability of readmission of an individual to a facility after a first admission, the system comprising:
an electronic medical record store comprising medical data elements for a pre-selected population;
a computer server at the computer healthcare system, the computer server coupled to the electronic medical record store and programmed to:
access the electronic medical record store to identify the medical data elements associated with a predetermined number of conditions that are associated with a first admission and at least one readmission to a facility for at least one individual over a predetermined time period;
analyze the medical data elements identified to establish a sample size;
perform a first sequential pattern analysis on the medical data elements to determine a correlation cluster between two or more conditions related to the first admission and the at least one readmission; and
in response to determining the correlation cluster, generate and communicate to a first user, via a user interface, a prediction for readmission for an individual based on the determined correlation cluster.

2. The system of claim 1, wherein the system further calculates a support percentage for the correlation cluster determined.

3. The system of claim 1, wherein the system further calculates a confidence percentage for the correlation cluster determined.

4. The system of claim 1, wherein the system further calculates a correlation coefficient for the correlation cluster determined.

5. The system of claim 1, wherein the system further calculates a regression coefficient and odds ratio after performing the first sequential pattern analysis.

6. The system of claim 1, wherein the correlation cluster and prediction for readmission are stored in a database.

7. The system of claim 1, wherein the system further receives an indication that a first individual has been admitted for at least one condition associated with the correlation cluster.

8. The system of claim 7, wherein based on a first admission of the first individual for the at least one condition associated with the correlation cluster occurring, generating one or more interventional treatment options to the first user via the user interface.

9. A dynamic system useful in a computer healthcare system to determine the probability of readmission of an individual to a facility after a first admission, the system comprising:
an electronic medical record store comprising medical data elements for a pre-selected population;
a computer server at the computer healthcare system, the computer server coupled to the electronic medical record store and programmed to:
access the electronic medical record store to identify the medical data elements associated with a predetermined number of conditions that are associated with a first admission and at least one readmission to a facility for at least one individual over a predetermined time period;
analyze the medical data elements identified to establish a sample size;
perform a first sequential pattern analysis on the medical data elements to determine a correlation cluster between two or more conditions related to the first admission and the at least one readmission for the at least one individual;
in response to determining the correlation cluster:
calculate one or more of a support percentage, confidence level, and correlation coefficient on the correlation cluster;
perform a multivariate logistic regression on the correlation cluster to determine a regression coefficient and an odds ratio; and
generate and communicate to a first user, via a user interface, a prediction for readmission for a first individual based on the determined correlation cluster.

10. The system of claim 9, wherein the system further receives a notification that the first individual has been admitted to the facility and diagnosed with one of the two or more conditions associated with the correlation cluster.

11. The system of claim 10, wherein the system further generates at least one interventional treatment option, via the user interface, to the first user to decrease the probability of readmission.

12. The system of claim 11, wherein the at least one interventional treatment is selected for the first individual by the first user.

13. The system of claim 12, wherein the at least one interventional treatment selected prevents a future readmission for the first individual.

14. The system of claim 13, wherein one or more clinical protocols are updated based on efficacy of the at least one interventional treatment option selected.

15. The system of claim 11, wherein the at least one interventional treatment option is rejected by the first user.

16. The system of claim 9, wherein the electronic medical record store is associated with at least one facility selected from: a hospital, an inpatient rehabilitation facility, and an acute care facility.

17. A computerized method carried out by a server for generating a correlation cluster and predicting future readmission for an individual, the method comprising:
accessing an electronic medical record store to identify medical data elements associated with a predetermined number of conditions that are associated with a first admission and at least one readmission to a facility for at least one individual over a predetermined time period;
analyzing the medical data elements identified to establish a sample size;
performing a first sequential pattern analysis on the medical data elements to determine a correlation cluster between two or more conditions associated with the first admission and the at least one readmission for the at least one individual;
calculating one or more of a support percentage, confidence level, and correlation coefficient on the correlation cluster;
performing a multivariate logistic regression on the correlation cluster to determine a regression coefficient and an odds ratio; and
generating and communicating a prediction for readmission for a first individual based on the determined correlation cluster.

18. The method of claim 17, further comprising receiving an indication that the first individual has been admitted to the facility with one or more of the two or more conditions associated with the correlation cluster.

19. The method of claim 18, further comprising providing a prediction to a user, via a user interface, of readmission for the first individual with one or more of the two or more conditions associated with the correlation cluster.

20. The method of claim 19, further comprising providing to the user, via the user interface, one or more interventional treatment options to decrease a risk of readmission of the first individual.
